(19) United States
(12) Patent Application Publication
(10) Pub. No.: US 2016/0194684 A1
(43) Pub. Date: Jul. 7, 2016

(54) METHOD FOR EXTRACTING AND PURIFYING NUCLEIC ACIDS AND BUFFERS USED

(71) Applicant: COMMISSARIAT À L'ÉNERGIE ATOMIQUE ET AUX ÉNERGIES ALTERNATIVES, Paris (FR)

(72) Inventor: Justine Lemonnier, GRENOBLE (FR)

(21) Appl. No.: 14/905,418

(22) PCT Filed: Jul. 16, 2014
(86) PCT No.: PCT/EP14/65311

§ 371 Date: Jan. 15, 2016

(30) Foreign Application Priority Data
Jul. 18, 2013 (FR) 13 57067

(51) Int. Cl. C12Q 1/68

(52) U.S. Cl. CPC C12Q 1/6806

(57) ABSTRACT

The present invention relates to a method for extracting and purifying at least one target nucleic acid contained in a sample, comprising the steps consisting of (a) putting the sample in contact with a lysis buffer optionally containing glycerol whereby a cell lysate is obtained; (b) putting the cell lysate obtained after step (a) in contact with magnetizable particles, under conditions allowing the capture of said at least one target nucleic acid by said particles; (c) washing said magnetizable particles obtained after step (b) with a washing buffer containing glycerol; (d) putting said washed particles obtained after step (c) in contact with an elution buffer, under conditions allowing release of said target nucleic acid in the elution buffer. The present invention also relates to said lysis and washing buffers.

### TECHNICAL FIELD

The present invention relates to the field of biology and more particularly to the field of molecular biology.

More particularly, the present invention proposes a method allowing extraction and purification of nucleic acids and this, for one (or several) subsequent analysis (analyses) of the amplification or sequencing type. The method according to the invention carried out from a sample containing nucleic acids uses buffers containing glycerol or magnetic or magnetizable particles or beads.

The present invention also relates to the buffers applied during this method and notably to the washing buffers and optionally to the lysis buffers.

### STATE OF THE PRIOR ART

In molecular biology, many studies and investigations are conducted on nucleic acids and this, in fields notably encompassing academic research; research and clinical or diagnostic analyses, with tests conducted on blood samples or cell or tissue biopsies; environmental monitoring; civil monitoring; food safety and notably quality control; criminal analyses on traces. . . . These investigations and studies all require the extraction and the purification beforehand of these nucleic acids.

Various methods are known at the present time for carrying out such extraction and purification. The diversity in these methods is the consequence of various criteria and parameters which have to be taken into account such as for example the target nucleic acid, the material containing it, the source organism of this material and the subsequent use of this target nucleic acid.

However, most of these methods have a sequence of steps consisting of (i) lyzing the material containing the target nucleic acid(s), (ii) removing the contaminating compounds notably of the protein type, present in the extract obtained at the end of step (i) and (iii) recovering the target nucleic acid(s).

The lysis step of the methods of the prior art should allow extraction of the nucleic acids of the material containing them while preserving their integrity. In other words, the lysis step should be sufficiently mild so as to preserve the nucleic acids while inhibiting or deactivating the enzymes which destroy these nucleic acids i.e. the nucleases. During this step, the lysis may be mechanical, chemical and/or enzymatic.

The steps (ii) and (iii) strictly speaking correspond to the purification of nucleic acids. These steps may implement one or several techniques selected from among:

extraction by solvents such as extraction by phenol-chloroform;

precipitation such as alcoholic precipitation or a precipitation with high salt concentrations;

a chromatographic technique such as filtration on a gel, chromatography with ion exchange, chromatography by adsorption or chromatography by affinity and

a centrifugation or an ultracentrifugation such as a centrifugation on cesium chloride.

Some of the above-disclosed techniques implement reagents, the handling of which requires precautions such as phenol. They may resort to a cumbersome apparatus such as an ultracentrifuge or too many steps making the extraction and purification process long. These different drawbacks make it difficult to automate the extraction and purification methods using such techniques.

In the techniques developed on the basis of adsorption or affinity chromatography, some implement magnetic or paramagnetic beads which have the capability of specifically adsorbing nucleic acids. This specific adsorption is related to the charge of the beads such as positively or negatively charged beads or to the presence, at the surface of the beads, of groups to which bind nucleic acids such as anti-DNA antibodies or oligonucleotides such as poly(thymine) oligonucleotides (or oligo(dT)).

Thus, the patent U.S. Pat. No. 6,855,499 in the name of Cortex Biochem Inc. aims to provide a simple method for isolating and purifying nucleic acids such as DNA (for “DesoxyriboNucleic Acid”), RNA (for “RiboNucleic Acid”) or PNA (for “Peptide Nucleic Acid”) from diverse samples such as blood, milk, seminal fluid, a tissue or bacterial cell lyzates and this without any centrifugation, without any alcohol and without preliminary preparation of the buffers. The technical solution proposed in this patent consists of using cellulose particles appearing as magnetic or paramagnetic particles, coated with cellulose or one of its derivatives. By adjusting the concentration of salts and of polyalkylene glycol in a solution containing nucleic acids and the cellulose particles, it is possible to obtain the binding of the nucleic acids on these particles. More particularly, the binding buffer described consists of polyethylene glycol with a molar mass of 8,000 g/mol (or PEG 8000) at 10% and 1.25 M NaCl. Further, the washing buffer applied also consists of 10% PEG 8000 and of NaCl but at 2.5 M. The kit PROMEGA MagaZorb® DNA mini prep is based on the procedure described in patent U.S. Pat. No. 6,855,499.

The state of the art is aware of other commercial kits using magnetic beads in procedures for extracting and purifying nucleic acids. For example, the Kit Silane Genomic DNA from Life Technologies (Ref 370-12D) is proposed for preparing nucleic acids from blood samples. In this kit, an alcohol of the ethanol or isopropanol type is used, which requires preparation of capture buffers and of washing buffers by adding thereto an alcohol before beginning extraction. In addition to the use of ethanol, this procedure has other drawbacks which are the incubation times of more than 3 min and 4 steps for washing the DNA-magnetic beads pellet.

The inventors realized that the commercial kits have extraction and purification yields which are not as good as when they are applied with other magnetic beads than those of the kit (see, in this respect, the example 5 of the experimental part hereafter). Also, the inventors set the goal of proposing a simple method in terms of steps, preparatory procedures and of reagents, in order to extract and purify nucleic acids, said method involving an adsorption or affinity chromatography step which may use any beads or particles, either magnetic or paramagnetic.

### DISCUSSION OF THE INVENTION

The inventors solved this technical problem and found a solution to the drawbacks of the methods of the prior art by proposing a complete method allowing extraction and purification of nucleic acids from various samples by applying lysis and washing buffers containing glycerol and this, by using magnetizable particles of various origins.

More particularly, the present invention proposes a method for extracting and purifying at least one target nucleic acid contained in a sample, comprising the successive steps consisting of:

a) putting the sample in contact with a lysis buffer whereby a cell lysate is obtained;

b) putting the cell lysate obtained after step (a) in contact with magnetizable particles, under conditions allowing capture of said at least one target nucleic acid by said particles;

c) washing said magnetizable particles obtained following step (b) with a washing buffer preferably containing glycerol; and

d) putting said washed particles obtained after step (c) in contact with an elution buffer, under conditions allowing the release of said target nucleic acid in the elution buffer.

By “nucleic acid”, is meant within the scope of the present invention, a chromosome; a gene; a regulator polynucleotide; a DNA, either single-stranded or double-stranded, genomic, chromosomic, chloroplastic, plasmid, mitochondrial, recombinant or complementary; a total RNA; a messenger RNA; a ribosomal RNA; a transfer RNA; a PNA; a portion or fragment thereof. The expression “nucleic acid” used in the present invention is equivalent to the following terms and expressions: “nucleotide molecule”, “polynucleotide”, “nucleotide sequence” and “polynucleotide sequence”. Further, in the present invention, the expression “target nucleic acid(s)” designates the nucleic acid(s) which one wishes to extract and purify by applying the claimed method.

It is obvious that the sample implemented within the scope of the method according to the present invention may be of highly diverse natures and sources. Generally, this is any sample from which one wishes to extract and purify the or some nucleic acids which it contains or with which it is contaminated.

Advantageously, this sample may be a biological fluid; a plant fluid such as sap, nectar and root exudate; a sample in a culture medium or in a biological cultivation reactor like a cell culture of superior eukaryotes, yeasts, fungi, bacteria, viruses or algae; a liquid obtained from an animal or plant tissue; an animal or plant tissue; one or several cells; a cell pellet; a sample from a food matrix, preferably diluted in a buffer; a sample from a chemical reactor; a sample in a water treatment plant; a sample in a composting plant; tap water, river water, pond water, lake water, sea water, swimming pool water, water from air-cooled towers or from underground; a sample from a liquid industrial effluent; waste water notably from intensive animal farming or industries in the chemical, pharmaceutical or cosmetic field; a sample from air filtration or from a coating; a sample on an object such as a fabric fragment, a piece of clothing, a sole, a shoe, a tool, a weapon, etc. . . . ; a pharmaceutical product; a cosmetic product; a perfume; a sample of earth or one of their mixtures.

Within the scope of the present invention, by <<sample>>is meant any type of collection of samples, for example by contact, scraping, drilling, cutting out, punching, milling, washing, rinsing, suction, pumping, etc. . . .

The biological fluid is advantageously selected from the group consisting of blood such as full blood or anti-coagulated full blood, blood serum, blood plasma, lymph, saliva, sputum, tears, sweat, sperm, urine, stools, milk, cerebrospinal liquid, interstitial liquid, isolated bone marrow fluid, mucus or fluid from the respiratory, intestinal or genitourinary tract, cell extracts, tissue extracts and organ extracts. Thus, the biological fluid may be any fluid naturally secreted or excreted from a human or animal body or any fluid recovered from a human or animal body, by any technique known to one skilled in the art such as an extraction, a sampling or a washing. The recovery and isolation steps of these different fluids from the human or animal body are carried out before implementing the method according to the invention.

The sample implemented within the scope of the present invention may contain, because of its nature or possibly following contamination, one or several cell(s) (either identical or different). By “cell”, is meant within the scope of the present invention, both a cell of the prokaryotic type and of the eukaryotic type. Among eukaryotic cells, the cell may be a yeast such as a yeast of the Saccharomyces or Candida genus, a plant cell or an animal cell such as a cell of mammals or insects. The cells of the prokaryotic type are bacteria which may be of the Gram positive type or Gram negative type bacteria. From among these bacteria, mention may be made as examples and in a non-exhaustive way, bacteria belonging to branches of spirochetes and chlamydiae, the bacteria belonging to the families of enterobacteria (such as Escherichia coli), streptococci (such as Streptococcus and in particular Streptococcus pneumoniae), micrococci (such as Staphylococcus), legionella, mycobacteria, bacillaceae and others.

Also, if one of the contemplated samples cannot be subjected to a method according to the present invention, for example because of its notably solid nature, of its concentration or of the elements which it contains such as solid residues, wastes, interfering suspension or molecules, this application and notably the extraction and purification method as defined hereafter further comprises a preliminary step for preparing the sample with optionally a putting into solution of the sample by techniques known to one skilled in the art such as filtration, precipitation, dilution, distillation, mixing, concentration, etc.

The step (a) of the method according to the present invention consists, like in the known extraction and purification methods of nucleic acids, of extracting the nucleic acids from the sample containing them while preserving their integrity and this notably by inhibiting or deactivating the enzymes which lyze the target nucleic acids.

Within the scope of the present invention, the lysis during step (a) is a chemical or enzymatic lysis step. Any lysis buffer for chemical and enzymatic lysis, known to one skilled in the art, may be used within the scope of the present invention. Advantageously, the lysis buffer implemented during step (a) of the method according to the invention contains glycerol.

More particularly, the lysis buffer implemented within the scope of the present invention is a buffer containing glycerol, at least one surfactant and at least one chaotropic agent.

The lysis buffer implemented within the scope of the present invention advantageously contains glycerol in an amount comprised between 1 and 40%, notably between 5 and 20%, in particular between 7 and 15% and, more particularly, of the order of 10% (i.e. 10%±2%) by mass relative to the total mass of the lysis buffer (i.e. a mass percentage). It is estimated that the presence of glycerol in the lysis buffer is not necessary, but advantageous, since it increases the total yield of the procedure.

The lysis buffer implemented within the scope of the present invention contains at least one surfactant, the latter participating in the chemical lysis. By “surfactant”, is meant a molecule including a lipophilic portion (apolar) and a hydrophilic portion (polar). Any surfactant able to solubilize cell membranes and membrane lipids may be used within the scope of the present invention.

Advantageously, this surfactant is selected from anionic surfactants, cationic surfactants, zwitterionic surfactants, amphoteric surfactants and non-ionic surfactants. The lysis buffer according to the invention may comprise several surfactants belonging to a same family of surfactants listed earlier (i.e. anionic, cationic, zwitterionic, amphoteric or non-ionic) or several surfactants belonging to at least two distinct families of surfactants.

As a reminder, anionic surfactants are surfactants for which the hydrophilic portion is negatively charged such as alkyl or aryl sulfonates, sulfates, phosphates, sulfosuccinates or sarcosinates associated with a counter ion like an ammonium ion (NH₄⁺), a quaternary ammonium such as tetrabutylammonium, and alkaline cations such as Na⁺, Li⁺ and K⁺. As anionic surfactants, for example it is possible to use tetraethylammonium paratoluenesulfonate, sodium dodecylsulfate (or SDS), sodium laurylsarcosinate (or sarcosyl), sodium palmitate, sodium stearate, sodium myristate, sodium di(2-ethylhexyl) sulfosuccinate, methylbenzene sulfonate and ethylbenzene sulfonate.

Cationic surfactants are surfactants for which the hydrophilic portion is positively charged, notably selected from quaternary ammoniums including at least one aliphatic C₄-C₂₂ chain associated with an anionic counter ion notably selected from boron derivatives such as tetrafluoroborate or halide ions such as F⁻, Br⁻, I⁻ or Cl⁻. As cationic surfactants, for example it is possible to use tetrabutyl-ammonium chloride, tetradecyl-ammonium chloride, tetradecyl-trimethyl-ammonium bromide (TTAB), cetyl-trimethyl-ammonium bromide (CTAB), octadecyl-trimethyl-ammonium bromide, hexadecyl-trimethyl-ammonium bromide, alkylpyridinium halides bearing an aliphatic chain and alkylammonium halides.

Zwitterionic surfactants are neutral compounds having formal electric charges of one unit and of opposite sign, notably selected from compounds having a C₅-C₂₀ alkyl chain generally substituted with a negatively charged function like a sulfate or a carboxylate and a positively charged function like an ammonium. As zwitterionic surfactants, mention may be made of sodium N,N dimethyl-dodecyl-ammoniumbutanate, sodium dimethyl-dodecyl-ammonium propanate and amino acids.

Amphoteric surfactants are compounds which behave both as an acid or as a base depending on the medium in which they are placed. As amphoteric surfactants, it is possible to use disodium lauroamphodiacetate and betaines like alkylamidopropylbetaine or laurylhydroxysulfobetaine.

Non-ionic surfactants, also known as neutral surfactants, are surfactants which do not have any group able to be ionized in water at a neutral pH or close to neutrality. Such surfactants are however amphipathic since they contain lipophilic entities and hydrophilic entities. The surfactant properties of non-ionic surfactants, notably hydrophilicity, are provided by non-charged functional groups such as an alcohol, an ether, an ester or further an amide, containing heteroatoms such as a nitrogen atom or an oxygen atom. Because of the low hydrophilic contribution of these functions, non-ionic surfactant compounds are most often polyfunctional. Any non-ionic surfactant known to one skilled in the art may be used within the scope of the present invention. As non-ionic surfactants, within the scope of the present invention it is possible to use alkyl, fatty alcohol, fatty amine, fatty acid, oxoalcohol or alkylphenol alkoxylates; alkyl, fatty alcohol, fatty amine, fatty acid, oxoalcohol or alkylphenol ethoxylates; monoesters (monolaurate, monomyristate, monostearate, monopalmitate, monooleate, etc), phospholipids and polyesters of fatty acids and of glycerol; polyglycerol fatty amides comprising on average from 1 to 5 moles of ethylene oxide; oxyethylene fatty acid esters of sorbitan including from 2 to 30 moles of ethylene oxide; monoesters (monolaurate, monomyristate, monostearate, monopalmitate, monooleate, etc) and polyesters of fatty acids and of sorbitan, polyoxyethylene sorbitan monoesters; polyols (surfactants derived from sugars) in particular glucose alkylates; surfactants derived from glucoside (sorbitol laurate) or from polyols; alkanolamides and mixtures thereof.

Within the scope of the present invention, the surfactant(s) implemented during step (a) are advantageously selected from non-ionic surfactants and anionic surfactants and mixtures thereof. Among the non-ionic surfactants implemented during step (a), the latter are advantageously selected from polyethoxylated ethers, polyoxyethylene sorbitan monoesters and mixtures thereof.

More particularly, the surfactant(s) implemented during step (a) is (are) selected from the group consisting of polyethylene glycol tert-octylphenyl ether (or Triton X-100®), polyoxyethylene sorbitan monolaurate 20 (or Tween 20®), sodium dodecylsulfate (or SDS), sodium laurylsarcosinate (or sarcosyl) and mixtures thereof. In a particular embodiment, the lysis buffer implemented during step (a) of the method according to the invention comprises a mixture of SDS and of Tween 20® and advantageously a mixture of SDS at 1% by mass and Tween 20® at 20% by mass relative to the total mass of the lysis buffer.

The lysis buffer implemented within the scope of the present invention contains at least one chaotropic agent, the latter also participating in chemical lysis. By “chaotropic agent”, is meant within the scope of the present invention, a salt which modifies the solubility of proteins and may cause their precipitation. Some of the chaotropic agents which may be used in the lysis buffer of the invention may further affect the three-dimensional structure of the proteins and denaturate them. Any chaotropic agent known to one skilled in the art may be used within the scope of the present invention.

Advantageously, the chaotropic agent implemented within the scope of the present invention is selected from the group consisting of sodium chloride (NaCl), ammonium sulfate ((NH₄)₂SO₄), sodium perchlorate (NaClO₄), sodium iodide (NaI), lithium chloride (LiCl), lithium perchlorate (LiClO₄), barium chloride (BaCl₂), cesium chloride (CsCl₂), potassium chloride (KCl), potassium iodide (KI), urea (CO(NH₂)₂), a guanidine salt such as guanidine thiocyanate (GuSCN) and mixtures thereof.

More particularly, the chaotropic agent implemented within the scope of the present invention is a mixture of sodium chloride and of guanidine thiocyanate. Typically, in the lysis buffer according to the invention, the sodium chloride is at a concentration greater than or equal to 0.5 M and notably of the order of 1 M (i.e. 1 M±0.2 M) and the guanidine thiocyanate is at a concentration comprised between 0.5 and 1 M and notably of the order of 0.85 M (i.e. 0.85 M±0.1 M).

The lysis buffer implemented within the scope of step (a) of the method according to the present invention may further comprise at least one element able to inhibit the DNAses and RNAses and/or at least one element able to destroy the disulfide bridges in the proteins or to prevent their re-formation. For this purpose, said lysis buffer may further comprise at least one element selected from among a thiol; a reducing agent such as β-mercapto-ethanol or dithiothreitol (DTT); ethylene diamine tetraacetic acid (EDTA) and a citrate. Also, if certain of the nucleic acids contained in the sample are not target nucleic acids, and consequently have to be removed such as for example, DNA or RNA, it is possible to add to the lysis buffer, compounds allowing chemical or enzymatic lysis of DNA or RNA. From among these compounds, mention may be made of sodium hydroxide (NaOH), an RNAse and a DNAse.

During the step (a) of the method according to the present invention, the amount of lysis buffer used is such that the sample/lysis buffer ratio (v/v) is comprised between 1/3 and 3, notably between 1/2 and 2 and, in particular, this ratio is equal to 1.

As the step (a) of the method according to the present invention is a lysis not only chemical but also enzymatic, it is necessary to add to the mixture formed with the sample and the lysis buffer, a protease and advantageously at least one endoprotease and this in order to achieve enzymatic digestion (or enzymatic proteolysis) of the proteins of the sample and to lead to the formation of more or less short peptide fragments from proteins contained in the sample. One skilled in the art is aware of various proteases, either specific or not, and notably various endoproteases, either specific or not, which may be used during step (a) of the method according to the invention.

Advantageously, the protease(s) implemented during step (a) of the method is(are) selected from the group consisting of proteinase K, subtilisin, pepsin, trypsin, chymotrypsin, thrombin, thermolysin, the HIV protease, elastase, the Xa factor, an endopeptidase with a thiol such as papain or a caspase, an endopeptidase specific of lysine (endoLysN), an endopeptidase specific of arginine (endoArgC) and an endopeptidase specific of glutamic acid (endoGluC). The protease which is more particularly implemented during step (a) of the method is a non-specific endoprotease and advantageously proteinase K. Still more particularly, this proteinase K is used at a concentration comprised between 0.1 and 5 mg/ml, notably between 0.4 and 2 mg/ml and in particular between 0.7 and 1.5 mg/ml of solution implemented during step (a). By “solution implemented during step (a)”, is meant the solution formed from the sample, the lysis buffer and the proteinase K.

The solution implemented during step (a) of the method according to the invention is homogenized manually or with a vortex and maintained at a temperature comprised between 40 and 65° C., notably between 50 and 60° C. and, in particular, of the order of 56° C. (i.e. 56° C.±3° C.) for a period comprised between 2 and 30 min notably between 5 and 20 min and in particular of the order of 10 min (i.e. 10 min±3 min). In this way and by action of the various elements added to the sample, a cell lysate containing the target nucleic acid(s) is obtained following step (a) of the method according to the present invention.

Step (b) consists of putting the cell lysate obtained during step (a) in contact with magnetizable particles able to reversibly capture, by adsorption or via specific interactions, nucleic acids.

By “magnetizable particles”, are meant particles having magnetic properties only when they are subjected to a magnetic field. Any magnetizable particles known to one skilled in the art may be used within the scope of the present invention.

The magnetizable particles implemented within the scope of the present invention are substantially spherical and their average diameter is advantageously comprised between 0.5 and 100 μm and notably between 1 and 50 μm. It should be noted that within the scope of the present invention, the expressions “magnetizable particle”, “(para)magnetic particle”, “magnetizable bead” and “(para)magnetic bead” are equivalent and may be used interchangeably.

The magnetizable particles implemented within the scope of the present invention comprise magnetic grains (or nanoparticles) and an organic or mineral matrix. Depending on the method used for preparing these magnetizable nanoparticles, it is possible to obtain various structures such as (i′) nanoparticles of the core/shell type with the core comprising magnetic grains and the shell formed by the organic or mineral matrix or (ii′) nanoparticles in which the magnetic grains are uniformly distributed in the organic or mineral matrix.

Advantageously, the magnetic grains (or nanoparticles) are in at least one material selected from the group consisting of iron, titanium, cobalt, zinc, copper, manganese or nickel metal oxides; magnetite; maghemite; hematite; manganese, nickel manganese-zinc ferrites and cobalt-nickel alloys.

The organic matrix of the magnetizable particles implemented within the scope of the present invention is a natural or synthetic organic polymer and, as an example, selected from the group consisting of albumin; cellulose and its derivatives notably as defined in patent U.S. Pat. No. 6,855,499; polyacrylates such as poly(acrylic acid) and poly(hydroxyethyl methacrylate); polyvinyl alcohol and polystyrene. Alternatively, when the magnetizable particles applied in the invention have a mineral matrix, the latter advantageously appears as a silica gel.

In a 1^{st} embodiment, the magnetizable particles implemented within the scope of the present invention may capture nucleic acids at their surface by adsorption. In this case, this capture may involve Van der Waals interactions, electrostatic interactions, hydrogen bonds or hydrophobic interactions. Advantageously, the adsorption of the nucleic acids on the magnetizable particles is accomplished by means of electrostatic interactions and of hydrogen bonds. For this purpose, the magnetizable particles have, at their surface, groups participating in such interactions or bonds, such as cationic or anionic groups. These groups are borne, intrinsically or by functionalization, by the matrix of the particles.

In a 2^{nd} embodiment, the magnetizable particles implemented within the scope of the present invention may capture nucleic acids at their surface by specific interactions. This embodiment requires functionalization, either covalent or not, of the surface of the particles with an element able to capture the target nucleic acids such as for example by hybridization. Such an element is advantageously selected from the group consisting of an oligonucleotide complementary to a specific sequence present in the target nucleic acids, a poly(thymine) oligonucleotide, streptavidin, biotin or an anti-DNA antibody.

One skilled in the art knows various methods for preparing optionally functionalized magnetizable particles as defined earlier. Alternatively, during step (b) of the method according to the invention, commercial magnetizable particles may be used. As illustrative and non-limiting examples of such particles, mention may be made of Silanol beads (CHEMICELL), MagPrep® HS beads (Merck SAS Estapor), Dynabeads® MyOne™ beads (Life Technologies), Dynabeads® Streptavidin beads (Life Technologies) and MagaZorb® beads (PROMEGA).

The ratio between the sample volume during step (a) and the volume of magnetizable particles implemented during step (b) is comprised between 1 and 50, notably between 5 and 25 and, in particular, of the order of 10 (i.e. 10±2).

The contacting during said step (b) is accomplished in the presence of a capture buffer and this in order to promote the reversible binding between the target nucleic acids contained in the cell lysate and the magnetizable particles. Advantageously, such a capture buffer comprises at least one solvent selected from the group consisting of 1,2-butanediol; 1,2-propanediol; 1,3-butanediol; 1-methoxy-2-propanol acetate; 3-methyl-1,3,5-pentanetriol; a dibasic ester such as DBE-2, DBE-3, DBE-4, DBE-5 or DBE-6; diethylene glycol monoethyl ether (DGME); diethylene glycol monoethyl ether acetate (DGMEA); ethyl lactate; ethylene glycol; poly(2-ethyl-2-oxazoline); a sodium salt of a copolymer of 4-styrene sulfonic acid and of maleic acid; tetraethylene glycol (TEG); tetraglycol; tetrahydrofurfuryl polyethylene glycol 200; triethylene glycol divinyl ether; anhydrous triethylene glycol and triethylene glycol monoethyl ether.

Advantageously, the capture buffer implemented during said step (b) of the method according to the invention comprises at least two solvents as defined earlier. In particular, the capture buffer implemented during said step (b) of the method according to the invention is a mixture of two solvents as defined earlier. More particularly, the capture buffer implemented during said step (b) of the method according to the invention is a mixture of TEG and of 1,2-butanediol and typically a mixture of 60% (v/v) of TEG and of 40% (v/v) of 1,2-butanediol.

The mixture implemented during step (b) of the method according to the invention containing the cell lysate, the magnetizable particles and the capture buffer as defined earlier is manually homogenized or with a vortex and maintained at a temperature comprised between 10 and 40° C., advantageously between 15 and 30° C. and, more particularly, at room temperature (i.e. 23° C.±5° C.) and this, for a period between 2 and 30 min, notably between 5 and 20 min and in particular of the order of 10 min (i.e. 10 min±3 min). Subsequent to step (b) of the method according to the invention, the magnetizable particles have captured the target nucleic acid(s).

Step (c) of the method according to the present invention consists of separating the magnetizable particles which have captured the target nucleic acids from the medium containing said particles at the end of (b) and, in particular, from other elements non-captured by the magnetizable particles (i.e. not bound to the latter) present in this medium. The latter is formed from the sample, the lysis buffer, the proteinase K and the capture buffer.

Advantageously, step (c) of the method according to the present invention consists of (c₁) submitting the magnetizable particles having captured the target nucleic acid(s) to a magnetic field, (c₂) removing the liquid phase i.e. the medium surrounding said magnetizable particles, (c₃) adding a washing buffer and (c₄) re-suspending said magnetizable particles in said washing buffer by suppressing the magnetic field, said steps (c₃) and (c₄) may be carried out one after the other or simultaneously.

The steps (c₁) to (c₄) are conventional steps in the various domains using magnetizable particles. The magnetic field (or magnetic force) may be applied, during step (c₁), by means of a magnet or a magnetic bench. Any technique aiming at removing a liquid may be used with the scope of step (c₂). As examples, this removal may be achieved by overturning, tapping, absorption or suction.

Step (c) of the method according to the present invention and the steps (c₁) to (c₄) may be repeated at least once. During these repetitions, the washing buffer applied during step (c) or (c₃) may be identical with or different from the washing buffer applied during the preceding step (c) or (c₃).

However, any washing buffer implemented in any of the steps (c) or (c₃) contains glycerol, a surfactant and optionally a chaotropic agent as defined earlier.

The washing buffer(s) implemented within the scope of the present invention contain(s) glycerol in an amount comprised between 1 and 40%, notably between 5 and 30%, and in particular between 7 and 25% by mass based on the total mass of the washing buffer.

The washing buffer(s) implemented within the scope of the present invention contain(s) a surfactant, in particular a surfactant from the family of ethylene glycols, for example a tetraethylene glycol (TEG) or a polyalkylene glycol. Any polyalkylene glycol may be used within the scope of the present invention. However, such a polyalkylene glycol is notably polyethylene glycol or polypropylene glycol and in particular polyethylene glycol (PEG). Polyethylene glycol in the washing buffer(s) has a molar mass of more than 1,000 g/mol, notably more than 2,000 g/mol, in particular, comprised between 4,000 and 10,000 g/mol and more particularly comprised between 6,000 and 8,000 g/mol. The washing buffer(s) implemented within the scope of the present invention contain(s) a surfactant from the family of ethylene glycols and notably tetraethylene glycol or polyethylene glycol in an amount comprised between 1 and 40%, notably between 5 and 30% and, in particular, between 7 and 25% by mass based on the total mass of the washing buffer.

The solvent of the washing buffer(s) is typically selected from Tris at a concentration comprised between 5 and 50 mM, Tris base at a concentration comprised between 5 and 50 mM, Tris/HCl at a concentration comprised between 5 and 50 mM, triethanolamine at a concentration comprised between 5 and 50 mM or one of their mixtures. The pH of the washing buffer is advantageously of the order of 8 (i.e. 8±0.5).

The ratio between the washing buffer volume implemented during each step (c) and the volume of implemented magnetizable particles during step (b) is comprised between 10 and 200, notably between 25 and 100 and in particular of the order of 50 (i.e. 50±5).

Step (c) of the method according to the present invention and notably the steps (c₁) and (c₄) are carried out at a temperature comprised between 10 and 40° C., advantageously between 15 and 30° C. and more particularly at room temperature (i.e. 23° C.±5° C.).

In a particular embodiment, the washing step is repeated twice. The washing buffer used during the 1^{st} washing step comprises glycerol, a surfactant like a surfactant from the family of ethylene glycols and a chaotropic agent as defined earlier and notably glycerol, PEG and a chaotropic agent or glycerol, TEG and a chaotropic agent as defined earlier, while the washing buffer used during the Ia 2^{nd} washing step comprises glycerol and a surfactant like a surfactant from the family of ethylene glycols as defined earlier and notably glycerol and PEG or glycerol and TEG as defined earlier. In the experimental part hereafter, particular examples of such washing buffers are given.

Step (d) of the method according to the present invention consists of re-suspending in solution the target nucleic acid(s) by detaching, by means of an elution buffer, the target nucleic acid(s) from the magnetizable particles. During this detachment, the non-covalent bonds and with low stability between the target nucleic acid(s) and the magnetizable particles are broken by the action of the elution buffer, optionally combined with the operating conditions of step (d) such as heating.

Advantageously, the step (d) of the method according to the present invention consists of (d₁) submitting the magnetizable particles to a magnetic field, (d₂) removing the washing buffer, (d₃) adding an elution buffer and (d₄) re-suspending said magnetizable particles in said elution buffer by suppressing the magnetic field, said steps (d₃) and (d₄) may be carried out one after the one or simultaneously.

The steps (d₁) to (d₄) are also conventional steps in various domains using magnetizable particles. The magnetic field (or magnetic force) may be applied, during step (d₁), by means of a magnet or a magnetic bench. Any technique aiming at removing a liquid may be used within the scope of step (d₂). As examples, this removal may be achieved by overturning, by tapping, by absorption or by suction.

The elution buffer implemented during step (d) or (d₃) may be any elution buffer known to one skilled in the art and adapted to the type of capture implemented during the preliminary step (b) i.e. adsorption or specific interactions. As an example of such an elution buffer, mention may be made of a buffer selected from Tris at a concentration comprised between 5 and 50 mM, from Tris base at a concentration comprised between 5 and 50 mM, of Tris/HCl at a concentration comprised between 5 and 50 mM, of triethanolamine at a concentration comprised between 5 and 50 mM or one of their mixtures. The pH of the elution buffer is advantageously of the order of 9 (i.e. 9±0.5).

The ratio between the elution buffer volume implemented during each step (d) and the volume of magnetizable particles applied during step (b) is comprised between 1 and 20, notably between 2 and 10 and, in particular of the order of 5 (i.e. 5±1).

Step (d) of the method according to the present invention and notably the step (d₄) may be carried out at a temperature comprised between 40 and 80° C., notably between 50 and 70° C. and in particular of the order of 60° C. (i.e. 60° C.±5° C.), the steps (d₁) to (d₃) may, on the other hand, be carried out a temperature comprised between 10 and 40° C., advantageously between 15 and 30° C. and, more particularly, at room temperature (i.e. 23° C.±5° C.).

Further, the step (d) of the method according to the present invention and notably the step (d₄) may be carried out with stirring and this for a duration comprised between 1 and 15 min, notably between 2 and 7 min and, in particular, of the order of 3 min (i.e. 3 min±1 min). This stirring is achieved by means of a magnetic stirrer and at a rate greater than or equal to 100 rpm, notably greater than or equal to 500 rpm and in particular, comprised between 1,000 and 1,500 rpm.

Once the step (d) of the method according to the invention is completed, it is easy to recover the elution buffer containing the target nucleic acid(s) by removing the magnetizable particles and this by applying a magnetic field.

The present invention also relates to lysis and washing buffers which may be applied in a method as defined earlier.

Thus, the present invention relates to a lysis buffer which may be implemented in a method as defined earlier, containing at least one surfactant, at least one chaotropic agent and glycerol as defined earlier. A particular example of such a lysis buffer is given in the experimental part hereafter.

Further, the present invention relates to a washing buffer which may be implemented in a method as defined earlier, containing glycerol, a surfactant, notably a surfactant from the family of ethylene glycols like TEG or a polyalkylene glycol and optionally a chaotropic agent as defined earlier and notably containing glycerol, TEG or PEG and optionally a chaotropic agent as defined earlier. Two particular examples of such a washing buffer are given in the experimental part hereafter.

The present invention also relates to a kit comprising at least one lysis buffer as defined earlier and at least one washing buffer as defined earlier. In a particular embodiment, the kit according to the invention comprises a lysis buffer and two washing buffers as given in the experimental part hereafter or a lysis buffer and the washing buffer no. 2 as given in the experimental part hereafter. The kit according to the present invention may further contain at least one element selected from the group consisting of (i″) a protease, notably an endoprotease and, in particular, proteinase K; (ii″) a capture buffer as defined earlier; (iii″) an elution buffer as defined earlier and (iv″) magnetizable particles as defined earlier.

The present invention further relates to the use of a lysis buffer of, a washing buffer and of a kit as defined earlier for extracting and purifying target nucleic acids in a sample, said extraction and purification using magnetizable particles.

In other words, the present invention relates to on the one hand a method for lyzing a sample consisting of putting said sample in contact with a lysis buffer according to the invention, and to on the other hand a method for washing magnetizable particles having captured at least one target nucleic acid consisting of putting said particles in contact with a washing buffer according to the invention.

Other features and advantages of the present invention will further become apparent to one skilled in the art upon reading the examples below given as an illustration and not as a limitation, with reference to the appended figures.

### SHORT DESCRIPTION OF THE DRAWINGS

FIG. 1 is a schematized illustration of the method according to the present invention.

FIG. 2 illustrates the purification percentage of the DNA versus the amount of glycerol contained in the lysis buffer and the washing buffers.

FIG. 3 shows the purification percentage of the DNA versus the amount of glycerol contained in the lysis buffer.

FIG. 4 shows the purification percentage of the DNA versus the amount of glycerol contained in the washing buffer No. 1.

FIG. 5 shows the purification percentage of the DNA versus the amount of glycerol contained in the washing buffer No. 2.

FIG. 6 shows the cycle threshold value or Ct for a supernatant obtained with the commercial PROMEGA method by using referenced magnetic beads which are the MagaZorb® beads or other non-referenced commercial beads like the Dynabeads® MyOne beads.

FIG. 7 shows the cycle threshold value or Ct for a supernatant obtained with the method according to the present invention by using various magnetic beads.

### DETAILED DISCUSSION OF PARTICULAR EMBODIMENTS

### EXAMPLE 1

### Variation of the Glycerol Concentration Present in the Washing and Lysis Buffers

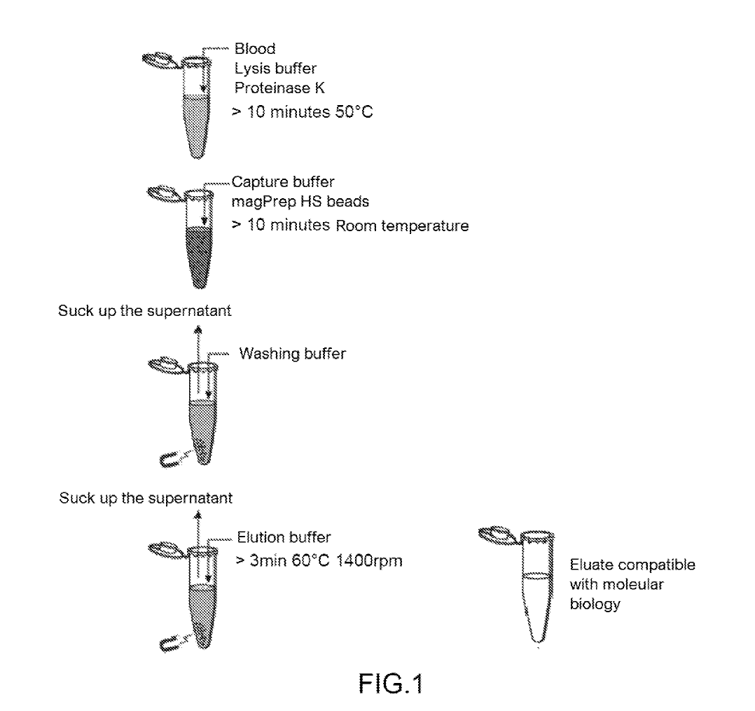

The procedure hereafter and schematized in FIG. 1 was used in order to study the effect of the glycerol concentration in the washing and lysis buffers. To this extent, for each independent test, a same concentration noted as “A %” corresponding to the glycerol mass based on the total mass of solution was used in the lysis and washing buffers, with A representing 0, 1, 10 or 20.

1. In a 1.5 mL tube containing 10,000 bacteria in a blood sample of 200 μL, said tube containing citrate or EDTA, add 200 μL of lysis buffer (SDS 1%, Tween20 20%, GuSCN 0.85 M, NaCl 1 M, glycerol A %), 28.6 μl proteinase K (20 mg/ml, Sigma—Cat. No. P2308, dissolved in 10 mM of Tris-HCl at pH 8);

2. Homogenize the tube with a vortex and incubate for 10 min at 56° C.;

3. Add 250 μL of capture buffer (TEG 60%, 1,2-butanediol 40%) and 20 μL of MagPrep HS beads (Merck SAS Estapor—Cat. No. Magprep HS 101899);

4. Homogenize the tube with a vortex and incubate for 10 min at room temperature;

5. Place the tube on a magnetic bench and let the pellet of magnetic particles-DNA settle and then remove the supernatant;

6. Wash the magnetic particles with 1 mL of washing buffer (2 M GuSCN, 10 mM Tris-HCl at pH 8, PEG8000 10%, glycerol A %);

7. Place the tube in a magnetic bench and let the pellet of magnetic particles-DNA settle and then remove the supernatant;

8. Wash the magnetic particles with 1 mL of washing buffer (10 mM Tris-HCl at pH 8, PEG8000 20%, glycerol A %);

9. Place the tube on a magnetic bench and let the pellet of magnetic particles-DNA settle and then remove the supernatant;

10. Elute the nucleic acids in 92 μl of elution buffer (10 mM Tris-HCl at pH 9) and stir with an Eppendorf—Thermomixer compact No. 5350 000.013 for 3 min at 1,400 rpm at 60° C.;

11. Place the tube on a magnetic bench and then recover the supernatant for carrying out different analyses.

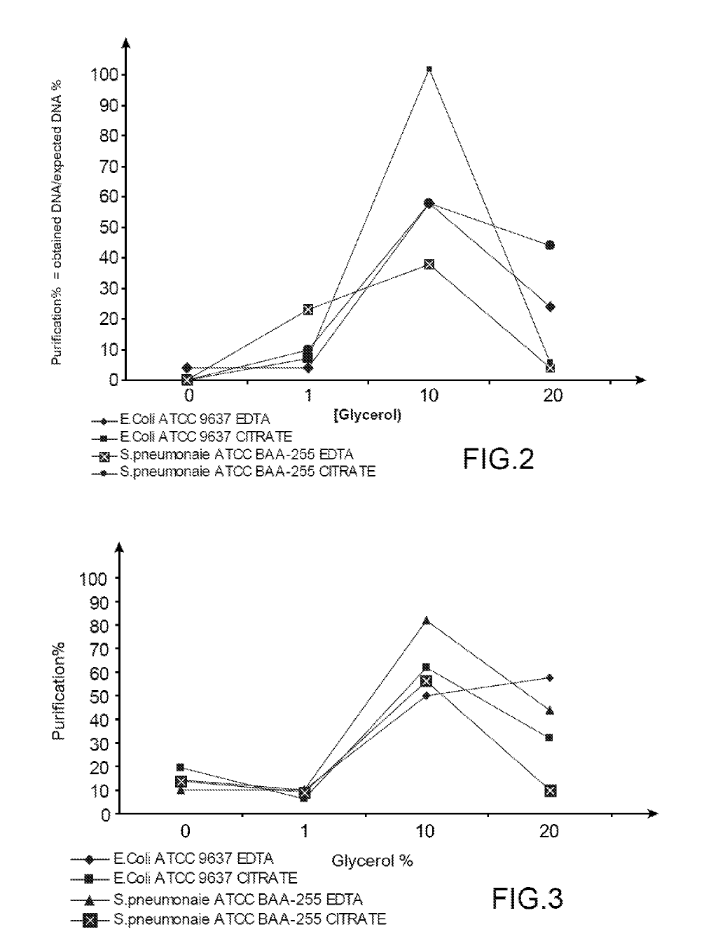

In the recovered supernatant, the DNA is analyzed by qPCR by using specific primers of the targeted pathogens. The amount of DNA present in the tube is thus obtained at the end of the procedure and the latter is compared with the total amount of DNA of the initial sample with, for the theoretical amount of DNA, the following calculation: 1 bacterium=1 DNA copy. The results are shown in FIG. 2.

In the absence of glycerol, no DNA is extracted. Extraction of DNA is obtained from an addition of 1% glycerol and this, for certain bacterial samples. The same applies for the procedure using 20% of glycerol in lysis and washing buffers. Better results as for the extraction for all the samples are obtained when the lysis buffer and the washing buffers contain 10% of glycerol.

### EXAMPLE 2

### Variation of the Glycerol Concentration Present in the Lysis Buffer

The procedure hereafter was applied for studying the effect of the glycerol concentration in the lysis buffer. To this extent, the concentration in the lysis buffer, noted as <<B %>> and corresponding to the glycerol mass based on the total mass of solution represents 0%, 1%, 10% or 20%.

1. In a 1.5 mL tube containing 10,000 bacteria in a blood sample of 200 μL, said tube containing citrate or EDTA, add 200 μL of lysis buffer (SDS 1%, Tween20 20%, GuSCN 0.85 M, NaCl 1 M, glycerol B %), 28.6 μl proteinase K (20 mg/ml, Sigma—Cat. No. P2308, dissolved in 10 mM of Tris-HCl at pH 8);

2. Homogenize the tube with a vortex and incubate for 10 min at 56° C.;

3. Add 250 μL of capture buffer (TEG 60%, 1,2-butanediol 40%) and 20 μL of MagPrep® HS beads (e.g. Merck SAS Estapor—Cat. No. Magprep HS 101899);

4. Homogenize the tube with a vortex and incubate for 10 min at room temperature;

5. Place the tube on a magnetic bench and let the pellet of magnetic particles-DNA settle and then remove the supernatant;

6. Wash the magnetic particles with 1 mL of washing buffer (2 M GuSCN, 10 mM Tris-HCl at pH 8, PEG8000 10%, glycerol 10%);

7. Place the tube on a magnetic bench and let the pellet of magnetic particles-DNA settle and then remove the supernatant;

8. Wash the magnetic particles with 1 mL of washing buffer (10 mM Tris-HCl at pH 8, PEG8000 20%, glycerol 20%);

9. Place the tube on a magnetic bench and let the pellet of magnetic particles-DNA settle and then remove the supernatant;

10. Elute the nucleic acids in 92 μl of elution buffer (10 mM Tris-HCl at pH 9) and stir with an Eppendorf™—Thermomixer® compact No. 5350 000.013 for 3 min at 1,400 rpm at 60° C.;

11. Place the tube on a magnetic bench and then recover the supernatant for conducting different analyses.

In the recovered supernatant, the DNA is analyzed by qPCR by using specific primers of the targeted pathogens. Thus the amount of DNA present in the tube at the end of the procedure is obtained and the latter is compared with the total amount of DNA of the initial sample with, for the theoretical amount of DNA, the following calculation: 1 bacterium=1 DNA copy. The results are shown in FIG. 3.

In the absence of glycerol in the lysis buffer and with washing buffers containing glycerol, DNA may be extracted. Adding 1% glycerol in the lysis buffer has very little effect on the extraction. On the other hand, the best results as for the extraction are obtained when the lysis buffer contains 10% of glycerol and are retained, for certain samples, with 20% glycerol in the lysis buffer. Thus, the presence of glycerol in the lysis buffer is not necessary; however it is preferably since it gives the possibility of increasing the total yield of the procedure.

### EXAMPLE 3

### Variation of the Glycerol Concentration Present in the Washing Buffer No. 1

The procedure hereafter was applied for studying the effect of the glycerol concentration in the washing buffer no. 1. For this study, the concentration in the washing buffer no. 1, noted as “C %” corresponding to the mass of glycerol based on the total mass of solution, represents 0%, 1%, 10% or 20%.

1. In a 1.5 mL tube containing 10,000 bacteria in a blood sample of 200 μL, said tube containing citrate or EDTA, add 200 μL of lysis buffer (SDS 1%, Tween20 20%, GuSCN 0.85 M, NaCl 1 M, glycerol 10%), 28.6 μl proteinase K (20 mg/ml, Sigma—Cat. No. P2308, dissolved in 10 mM of Tris-HCl at pH 8);

2. Homogenize the tube with a vortex and incubate for 10 min at 56° C.;

3. Add 250 μL of capture buffer (TEG 60%, 1,2-butanediol 40%) and 20 μL of MagPrep® HS beads (Merck SAS Estapor®—Cat. No. Magprep HS 101899);

4. Homogenize the tube with a vortex and incubate for 10 min at room temperature;

5. Place the tube on a magnetic bench and let the pellet of magnetic particles-DNA settle and then remove the supernatant;

6. Wash the magnetic particles with 1 mL of washing buffer (2 M GuSCN, 10 mM Tris-HCl at pH 8, PEG8000 10%, glycerol C %);

7. Place the tube on a magnetic bench and let the pellet of magnetic particles-DNA settle and then remove the supernatant;

8. Wash the magnetic particles with 1 mL of washing buffer (10 mM Tris-HCl at pH 8, PEG8000 20%, glycerol 20%);

9. Place the tube on a magnetic bench and let the pellet of magnetic particles-DNA settle and then remove the supernatant;

10. Elute the nucleic acids in 92 μl of elution buffer (10 mM Tris-HCl at pH 9) and stir with an Eppendorf™—Thermomixer® compact No. 5350 000.013 for 3 min at 1,400 rpm at 60° C.;

11. Place the tube on a magnetic bench and then recover the supernatant for conducting different analyses.

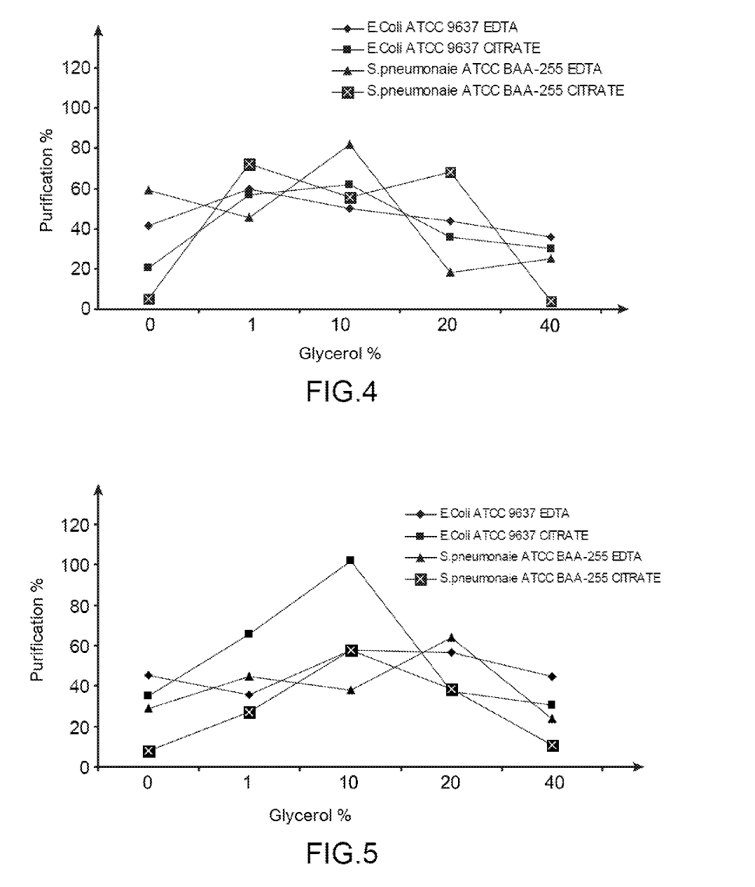

In the recovered supernatant, the DNA is analyzed with qPCR by using specific primers of the targeted pathogens. Thus the amount of DNA present in the tube is obtained at the end of the method and the latter is compared with the total amount of DNA of the initial sample with, for the theoretical amount of DNA, the following calculation: 1 bacterium=1 DNA copy. The results are shown in FIG. 4.

When compared with DNA extraction with 0% glycerol in the washing buffer no. 1, an increase in the DNA extraction may be observed as soon as 1% of glycerol is added into this buffer and is maintained for certain samples up to a 40% content of glycerol.

### EXAMPLE 4

### Variation of the Glycerol Concentration Present in the Washing Buffer No. 2

The procedure hereafter was applied for studying the effect of the glycerol concentration in the washing buffer no. 1. For this study, the concentration in the washing buffer no. 2, noted as “D %” corresponding to the mass of glycerol based on the total mass of solution represents 0%, 1%, 10% or 20%.

1. In a 1.5 mL tube containing 10,000 bacteria in a blood sample of 200 μL, said tube containing citrate or EDTA, add 200 μL of lysis buffer (SDS 1%, Tween20 20%, GuSCN 0.85 M, NaCl 1 M, glycerol 10%), 28.6 μl proteinase K (20 mg/ml, Sigma—Cat. No. P2308, dissolved in 10 mM of Tris-HCl at pH 8);

2. Homogenize the tube with a vortex and incubate for 10 min at 56° C.;

3. Add 250 μL of capture buffer (TEG 60%, 1,2-butanediol 40%) and 20 μL of MagPrep® HS beads (Merck SAS Estapor®—Cat. No. Magprep® HS 101899);

4. Homogenize the tube with a vortex and incubate for 10 min at room temperature;

5. Place the tube on a magnetic bench and let the pellet of magnetic particles-DNA settle and then remove the supernatant;

6. Wash the magnetic particles with 1 mL of washing buffer (2 M GuSCN, 10 mM Tris-HCl at pH 8, PEG8000 10%, glycerol 10%);

7. Place the tube on a magnetic bench and let the pellet of magnetic particles-DNA settle and then remove the supernatant;

8. Wash the magnetic particles with 1 mL of washing buffer (10 mM Tris-HCl at pH 8, PEG8000 20%, glycerol D %);

9. Place the tube on a magnetic bench and let the pellet of magnetic particles-DNA settle and then remove the supernatant; 10. Elute the nucleic acids in 92 μl of elution buffer (10 mM Tris-HCl at pH 9) and stir with an Eppendorf—Thermomixer compact No. 5350 000.013 for 3 min at 1,400 rpm at 60° C.;

11. Place the tube on a magnetic bench and then recover the supernatant for conducting various analyses.

In the recovered supernatant, the DNA is analyzed with qPCR by using specific primers of the targeted pathogens. The amount of DNA present in the tube at the end of the method is thus obtained and the latter is compared with the total amount of DNA of the initial sample with, for the theoretical amount of DNA, the following calculation: 1 bacterium=1 DNA copy. The results are shown in FIG. 5.

When compared with the DNA extraction with 0% glycerol in the washing buffer no. 2, an increase in the DNA extraction may be observed as soon as 1% glycerol is added into this buffer and is maintained for certain samples up to a 40% content of glycerol.

### EXAMPLE 5 (COMPARATIVE)

### Procedure for Commercial Extraction PROMEGA Applied with Different Beads

The commercial PROMEGA procedure below was implemented with magnetic beads adapted to this procedure, i.e. MagaZorb® beads (PROMEGA—Cat. No. MB1004) but also with other commercial beads not referenced i.e. Dynabeads® MyOne™ magnetic beads (Life Technologies—Cat. No. Dynabeads® MyOne™ Silane 37002D).

1. In a 1.5 mL tube containing 10,000 bacteria in a blood sample of 200 μL, said tube containing citrate, add 200 μL of lysis buffer PROMEGA, 20 μl of proteinase K PROMEGA;

2. Homogenize the tube with a vortex and incubate for 10 min at 56° C.;

3. Add 500 μL of capture buffer PROMEGA and 20 μL of magnetic beads:
MagaZorb® (PROMEGA—Cat. No. MB1004);
Dynabeads® MyOne™ (Life Technologies—Cat. No. Dynabeads® MyOne™ Silane 37002D);

4. Homogenize the tube with a vortex and incubate for 10 min at room temperature;

5. Place the tube on a magnetic bench and let the pellet of magnetic particles-DNA settle and then remove the supernatant;

6. Wash the magnetic particles with 1 mL of washing buffer PROMEGA;

7. Place the tube on a magnetic bench and let the pellet of magnetic particles-DNA settle and then remove the supernatant;

8. Wash the magnetic particles with 1 mL of washing buffer PROMEGA;

9. Place the tube on a magnetic bench and let the pellet of magnetic particles-DNA settle and then remove the supernatant;

10. Elute the nucleic acids in 100 μl of elution buffer PROMEGA and incubate for 10 min at room temperature;

11. Place the tube on a magnetic bench and then recover the supernatant for quantitative PCR analyses.

As a reminder, in quantitative PCR, the amount of amplicons produced at each cycle is tracked by measuring fluorescence. When the latter becomes statistically and significantly greater than the background noise, the corresponding amplification cycle is defined as the cycle threshold or Ct. There exists a linear relationship between the amount of nucleic matrices in the obtained supernatant following the extraction/purification procedure and the Ct obtained with this supernatant. In other words, the higher the Ct, the lower is the amount of matrices in the supernatant and therefore the lower is the extraction/purification yield. For analyzing the Ct with PCR, it is important to note that a difference of less than 1 Ct is not significant.

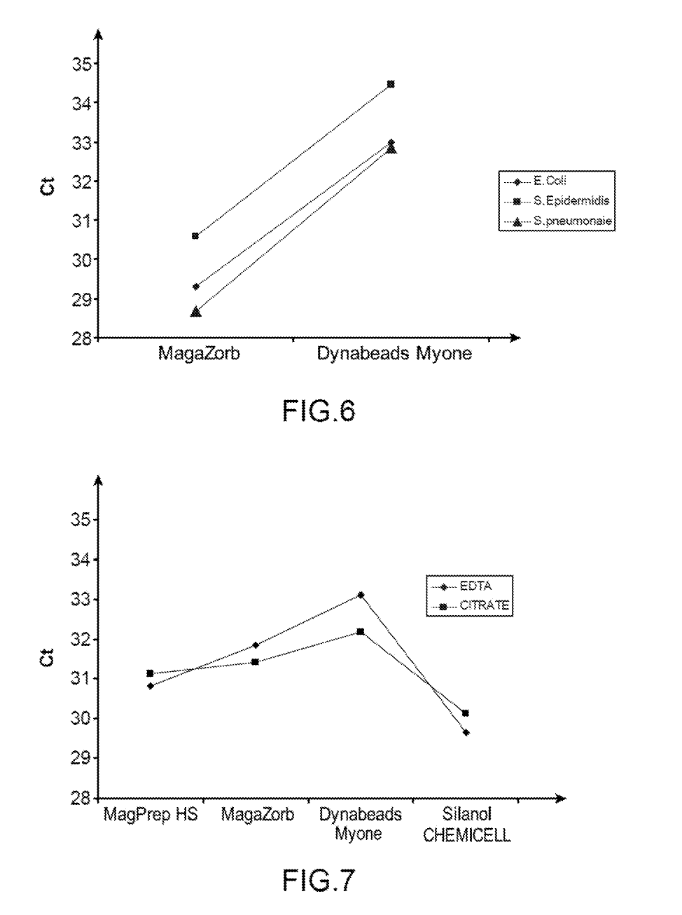

The results of the Cts of the supernatants obtained with the PROMEGA procedure using referenced magnetic beads (MagaZorb®, PROMEGA) or non-referenced commercial beads (Dynabeads® MyOne™, Life Technologies) are given in FIG. 6. The obtained eluate contains the DNA of three pathogens (E. coli, S. epidermidis, S. pneumoniae). FIG. 6 illustrates the Cts obtained for each pathogen.

The loss of 3 Cts for the PROMEGA procedure when non-referenced beads are used shows that this procedure is adapted to referenced beads, i.e. the MagaZorb® beads. It should be noted that this was experimentally verified (NMR analysis) that the lysis and washing buffers of the PROMEGA procedure did not contain any glycerol.

### EXAMPLE 6

### The Method According to the Invention with Magnetic Beads from Different Suppliers

In this example, different commercial magnetic beads were used following the method of the invention described hereafter:

1. In a 1.5 mL tube containing 10,000 E. coli bacteria in a blood sample of 200 μL, said tube containing citrate or EDTA, add 200 μL of lysis buffer (SDS 1%, Tween20 20%, GuSCN 0.85 M, NaCl 1 M, glycerol 10%), 28.6 μl of proteinase K (20 mg/ml, Sigma—Cat. No. P2308, dissolved in 10 mM of Tris-HCl at pH 8);

2. Homogenize the tube with a vortex and incubate for 10 min at 56° C.;

3. Add 250 μL of capture buffer (TEG 60%, 1,2-butanediol 40%) and 20 μL of magnetic beads:

MagPrep® HS (Merck SAS Estapor®—Cat. No. Magprep HS 101899);

Dynabeads® MyOne™ (Life Technologies—Cat. No. Dynabeads® MyOne™ Silane 37002D);

MagaZorb® (PROMEGA—Cat. No. MB1004);

Silanol (CHEMICELL—Cat. No. SiMAG-Silanol 1101-1);

4. Homogenize the tube with a vortex and incubate for 10 min at room temperature;

5. Place the tube on a magnetic bench and let the pellet of magnetic particles-DNA settle and then remove the supernatant;

6. Wash the magnetic particles with 1 mL of washing buffer (2 M GuSCN, 10 mM Tris-HCl at pH 8, PEG8000 10%, glycerol 10%);

7. Place the tube on a magnetic bench and let the pellet of magnetic particles-DNA settle and then remove the supernatant;

8. Wash the magnetic particles with 1 mL of washing buffer (10 mM Tris-HCl at pH 8, PEG8000 20%, glycerol 20%);

9. Place the tube on a magnetic bench and let the pellet of magnetic particles-DNA settle and then remove the supernatant;

10. Elute the nucleic acids in 92 μl of elution buffer (10 mM Tris-HCl at pH 9) and stir with an Eppendorf®—Thermomixer™ compact No. 5350 000.013 for 3 min at 1,400 rpm at 60° C.;

11. Place the tube on a magnetic bench and then recover the supernatant for conducting different analyses.

FIG. 7 shows the obtained Cts, corresponding to the DNA extracted from E. Coli, by applying the method according to the present invention with commercial magnetic beads of different origins. With the buffers containing glycerol, better homogeneity in the Cts is obtained, showing that these buffers are compatible with a larger variety of beads. Thus, the results shown demonstrate the possibility of using different magnetic beads with the method according to the present invention.

1. A method for extracting and purifying at least one target nucleic acid contained in a sample, comprising the successive steps:
a) putting the sample in contact with a lysis buffer whereby a cell lysate is obtained;
b) putting the cell lysate obtained after step (a) in contact with magnetizable particles, under conditions allowing capture of said a least one target nucleic acid by said particles;
c) washing said magnetizable particles obtained subsequently to step (b) with a washing buffer containing glycerol; and
d) putting said washed particles obtained after step (c) in contact with an elution buffer, under conditions allowing the release of said target nucleic acid in the elution buffer.

2. The method according to claim 1, wherein the lysis buffer implemented during said step (a) contains glycerol.

3. The method according to claim 1, wherein said nucleic acid is selected from the group consisting of a chromosome; a gene; a regulator polynucleotide; a DNA, either a single-stranded or double-stranded, genomic, chromosomal, chloroplastic, plasmid, mitochondrial, recombinant or complementary; a total RNA; a messenger RNA; a ribosomal RNA; a transfer RNA; a PNA; a portion and fragment thereof.

4. The method according to claim 1, wherein said sample is selected from the group consisting of a biological fluid; a plant fluid; a sample from a culture medium or from a biological culture reactor; a liquid obtained from an animal or plant tissue; an animal or plant tissue; one or several cells; a cell pellet; a sample from a food matrix; a sample from a chemical reactor; a sample from a water treatment plant; a sample from a compositing facility; tap water, river water, pond water, lake water, sea water, swimming pool water, water from air-cool towers, water from underground; a sample from a liquid industrial effluent; waste water; a sample from air filtration or from a coating; a sample from an object; a pharmaceutical product; a cosmetic product; a perfume; a sample of earth or one of their mixtures.

5. The method according to claim 1, wherein said lysis buffer is a buffer containing glycerol, at least one surfactant and at least one chaotropic agent.

6. The method according to claim 5, wherein the glycerol in said lysis buffer is in an amount comprised between 1 and 40%, by mass relative to the total mass of the lysis buffer.

7. The method according to claim 5, wherein said surfactant is selected from non-ionic surfactants and anionic surfactants and mixtures thereof.

8. The method according to claim 5, wherein said chaotropic agent is selected from the group consisting of sodium chloride (NaCl), ammonium sulfate ((NH₄)₂SO₄), sodium perchlorate (NaClO₄), sodium iodide (NaI), lithium chloride (LiCl), lithium perchlorate (LiClO₄), barium chloride (BaCl₂), cesium chloride (CsCl₂), potassium chloride (KCl), potassium iodide (KI), urea (CO(NH₂)₂), a guanidine salt and mixtures thereof.

9. The method according to claim 1, wherein during said step (a), at least one protease and at least one endoprotease is added to the mixture consisting of the sample and the lysis buffer.

10. The method according to claim 1, wherein during said step (b), said contacting is accomplished in the presence of a capture buffer, the latter comprising at least one solvent selected from the group consisting of 1,2-butanediol; 1,2-propanediol; 1,3-butanediol; 1-methoxy-2-propanol acetate; 3-methyl-1,3,5-pentanetriol; a dibasic ester selected from DBE-2, DBE-3, DBE-4, DBE-5 or DBE-6; diethylene glycol monoethyl ether (DGME); diethylene glycol monoethyl ether acetate (DGMEA); ethyl lactate; ethylene glycol; poly(2-ethyl-2-oxazoline); a sodium salt of a copolymer of 4-styrene-sulfonic acid and of maleic acid; tetraethylene glycol (TEG); tetraglycol; tetrahydrofurfuryl polyethylene glycol 200; triethylene glycol divinylether; anhydrous triethylene glycol and triethylene glycol mono-ethyl ether.

11. The method according to claim 1, wherein the washing buffer implemented during said step (c) contains glycerol, a surfactant, and optionally a chaotropic agent.

12. The method according to claim 11, wherein the glycerol is in an amount comprised between 1 and 40%, by mass relative to the total mass of the washing buffer.

13. The method according to claim 11, wherein said surfactant is polyethylene glycol having a molar mass greater than 1,000 g/mol, and/or is found in said washing buffer in an amount comprised between 1 and 40%, by mass relative to the total mass of the washing buffer.

14. The method according to claim 1, wherein said step (c) is repeated at least once.

15. The method according to claim 1, wherein said elution buffer is a buffer selected from among Tris at a concentration comprised between 5 and 50 mM, Tris base at a concentration comprised between 5 and 50 mM, Tris/HCl at a concentration comprised between 5 and 50 mM, triethanolamine at a concentration comprised between 5 and 50 Mm and a mixture thereof or one of their mixtures, wherein the pH of said elution buffer is 9±0.5.

16. A lysis buffer which may be implemented in a method as defined in claim 1, containing at least one surfactant, at least one chaotropic agent and glycerol, wherein said chaotropic agent is a mixture of sodium chloride and guanidine thiocyanate.

17. A washing buffer which may be implemented in a method as defined in claim 1, containing glycerol, a surfactant from the family of ethylene glycols and optionally a chaotropic agent.

18. A kit of elements comprising:
at least one lysis buffer containing at least one surfactant, at least one chaotropic agent and glycerol, wherein said chaotropic agent is a mixture of sodium chloride and guanidine thiocyanate and
at least one washing buffer containing glycerol, a surfactant from the family of ethylene glycols and optionally a chaotropic agent.

19. The method according to claim 5, wherein the glycerol in said lysis buffer is in an amount comprised between 5 and 20% by mass relative to the total mass of the lysis buffer.

20. The method according to claim 11, wherein the surfactant is tetraethylene glycol or a polyalkylene glycol.
